(19) United States
(12) Patent Application Publication
(10) Pub. No.: US 2019/0358198 A1
(43) Pub. Date: Nov. 28, 2019

(54) USE OF OVATODIOLIDE FOR PREPARING A COMPOSITION FOR INHIBITING PROTEIN SYNTHESIS OF GASTRIC HELICOBACTER PYPORI

(71) Applicant: Syi Biotechnology Co.,Ltd., Taichung City (TW)

(72) Inventors: Hsiu-Man Lien, Taichung City (TW); Chia-Chang Chen, Taichung City (TW); Chih-Ho Lai, New Taipei City (TW); Sheau-Jiun Chang, Taichung City (TW); Hui-Yu Wu, New Taipei City (TW)

(21) Appl. No.: 15/987,438

(22) Filed: May 23, 2018

(51) Int. Cl. A61K 31/365; A61P 31/04

(52) U.S. Cl. CPC A61K 31/365; A61P 31/04

(57) ABSTRACT

A use of ovatodiolide for preparing a composition used for inhibiting protein synthesis of gastric Helicobacter pylori, wherein the ovatodiolide achieves an effect of inhibiting gastric Helicobacter pylori by inhibiting the expression of 30S ribosomes RpsB of gastric Helicobacter pylori and further inhibiting its protein synthesis.

### BACKGROUND OF THE INVENTION

### Field of the Invention

The present invention relates to the natural medicinal herbs field of ovatodiolide and more particularly to the use of ovatodiolide for inhibiting drug-resistant gastric Helicobacter pylori.

### Description of the Related Art

Helicobacter pylori is a microaerophilic Gram-negative bacteria, while human being is the only host. One of the most common Helicobacter pylori infections for the patent is the mucous membrane of the stomach and the duodenum. Epidemiological studies have found that the correlation between Helicobacter pylori and gastric cancer is up to 70% (Marshall B. 2002). Many studies have pointed out that Helicobacter pylori infection may cause gastrointestinal diseases, such as gastric ulcer, chronic gastritis, and even gastric adenocarcinoma (Marshall B. J. and Warren J. R., 1984, NIH Consensus Conference 1994 and IARC Working Group on the Evaluation of Carcinogenic Risks to Humans. 1994). Therefore, eradicating the Helicobacter pylori can reduce the probability of gastric cancer.

Currently, triple therapy is the main method for eradicating Helicobacter pylori. Besides, mono therapy, dual therapy and quadruple therapy are also available. The mono therapy is the use of a single agent treatment, such as antibiotics and bismuth, etc. The dual therapy takes 2 weeks to combine use one type of antibiotic with bismuth or proton pump inhibitor (PPI). The triple therapy takes 1-2 weeks for bismuth or proton pump inhibitor plus with two types of antibiotics. The quadruple therapy takes 1 week for bismuth or proton pump inhibitor plus two types of antibiotics, but the use of the agents is complex and easy to interact with each other and produce new side effects.

Currently, antibiotics commonly used on the market for eradicating Helicobacter pylori include clarithromycin, amoxicillin, flurithromycin, tetracycline and metronidazole. The role of proton pump inhibitor in Helicobacter pylori is mainly to inhibit the formation of gastric acid and the effect of bacteriostasis, commonly used include omeprazole and lansoprazole. Bismuth has the effects of killing Helicobacter pylori and protecting gastric parietal cells. Bismuth (III) and bismuth (V) are commonly used. Antibiotics are currently the most effective drugs for eradicating infection of Helicobacter pylori. However, the drug resistance caused by improper use of antibiotics has become the main reason of the failure of Helicobacter pylori eradication. In order to solve the difficulty of treatment caused by drug resistance, a non-antibiotic therapy has been studied in recent years that chemical compounds extracted from natural medicinal herbs can effectively treat Helicobacter pylori (Lee S. Y., et al. 2008). Anisomeles indica (also known as Indian Epimerdei or Kabling-parang) belonging to the Labiatae family is a perennial herbaceous plants grown in subtropical region, and commonly used for curing related diseases of gastroenteritis and immunodeficiency, previously have discovered that its extracts can effectively inhibit inflammation response and tumor proliferation (Rao Y. K., et al. 2009, Hsieh S. C., et al. 2008). Additionally, the medical documents (Lien H. M., et al. 2013 and Rao Y. K., et al. 2012) have been disclosed that Anisomeles indica extract has the effect of inhibiting Helicobacter pylori. However, its inhibition mechanism and the efficacy of inhibiting drug resistance to Helicobacter pylori are still unclear. Therefore, the mechanism of inhibition of Helicobacter pylori by Anisomeles indica is an urgent problem to be solved in this field.

### SUMMARY OF THE INVENTION

In view of the above, the inventor is deeply aware of the deficiencies and defects of the existing technology, and is eager to improve and innovate. After many years of research, the inventor has successfully developed an ovatodiolide extracted from Anisomeles indica for inhibiting the protein synthesis of gastric Helicobacter pylori.

In order to achieve the above-mentioned objects, the present invention provides a use of ovatodiolide for preparing a composition used for inhibiting protein synthesis of gastric Helicobacter pylori, wherein the ovatodiolide achieves an effect of inhibiting gastric Helicobacter pylori by inhibiting protein synthesis.

Gastric Helicobacter pylori refers to multidrug resistant gastric Helicobacter pylori or Helicobacter pylori type strain. The multidrug resistant gastric Helicobacter pylori refers to HP v633 or HP v1354. The Helicobacter pylori type strain is H. pylori 26695.

The protein synthesis is through the action of 30S ribosome RpsB.

An effective dose of the ovatodiolide inhibiting the gastric Helicobacter pylori is between 16 mg and 1642 mg; a preferred dose is between 82 mg and 821 mg; and an optimal dose is between 164 mg and 329 mg.

The composition further comprises pharmaceutically acceptable carrier, vehicle and thinner; a dosage form is selected from a group composed of solution, suspension, emulsion, powder, pastille, pellet, syrup, troche, tablet, chewing gum, jatex and capsule.

The composition can be further made into fluid milk products, concentrated milk, yogurt, sour milk, frozen yogurt, lactic acid bacteria-fermented beverages, milk powder, ice cream, cream cheeses, hard cheeses, soy milk, fermented soy milk, vegetable-fruit juices, juices, sports drinks, confectionery, jelly, candies, infant foods, health foods, animal feeds, Chinese medicinal herbs compositions and dietary supplements.

### BRIEF DESCRIPTION OF THE DRAWINGS

FIG. 1 is a graph of amount of protein generation of Helicobacter pylori inhibited by ovatodiolide; and

FIG. 2 is an expression of 30S ribosomal subunits analyzed by western blot analysis.

### DETAILED DESCRIPTION OF THE INVENTION

All the technical and scientific terms mentioned in the specification are meanings that can be commonly understood by professionals of the field unless otherwise defined.

The terms, carrier and vehicle, in the specification refer to non-toxic chemical compounds or medications, which have the effect of assisting cells or tissues to absorb medicines.

The above-mentioned terms can be aromatics, buffers, binders, colorants, disintegrants, thinners, emulsifiers, extenders, flavor-improving agents, gellants, glidants, antiseptics, skin-penetration enhancers, solubilizers, stabilizers, suspending agents, sweeteners, tonicity agents, viscosity-enhancing agents, or random compositions of the above.

The term, effective dose, in the specification refers to an adequate amount of chemical compounds or medicines that one or multiple disease symptoms or physiological conditions are relieved after a person took the medicine; resulted in the reducing and/or abirritation of signs, symptoms or causes of diseases, or other purposeful changes of the physiological systems. For example, therapeutically effective dose includes a dose of chemical compounds provided by the present invention that can clinically and remarkably reduce the disease symptoms. An effective value of an appropriate effective dose depends on general pharmaceutical techniques, such as dose escalation methods.

The composition used in the present invention can be further added with one edible material for preparing as a food product or health care product. Wherein the edible material comprises but is not limited to: water, fluid milk products, milk, concentrated milk, fermented milk, such as yogurt, sour milk, frozen yogurt, lactic acid bacteria-fermented beverages; milk powder, ice cream; cream cheeses, hard cheeses, soy milk, fermented soy milk, vegetable-fruit juices, juices, sports drinks, confectionery, jelly, candies, infant foods, health foods, animal feeds, Chinese medicinal herbs and dietary supplements.

The above-mentioned composition used in the present invention can be dietary supplements, which can be provided as the following ways for a person who takes it: mixed with an appropriate potable fluid, such as water, yogurt, milk or juice; or mixed with solid or fluid foods. In the specifications, the forms of dietary supplements can be pastille, pellet, capsule, lozenge, granule, powder, suspending agent, sachet, soft pastille, candy, bar, syrup and corresponding given forms; generally in the form of dose unit and is manufactured by conventional methods for preparing dietary supplements.

The following embodiments are merely for exemplifications. Doses can be changed according to variations, and are not limited to the activity of chemical compounds being used, diseases being treated or physiological conditions, ways of administration, individual needs and requirements, severity of diseases and judgments of doctors.

### Embodiment 1: Minimum Bacteriostatic Concentrations of Ovatodiolide for Inhibiting Helicobacter pylori

Different concentrations of ovatodiolide disks are used to cultivate with Helicobacter pylori for two days respectively. Then, the minimum concentrations for inhibiting Helicobacter pylori are determined by using agar-well diffusion. The results are used for analyzing the minimum concentration of ovatodiolide for inhibiting the growth of Helicobacter pylori type strain (H. pylori 26695). Table 1 shows the minimum bacteriostatic concentrations for inhibiting Helicobacter pylori type strain (H. pylori 26695). Based on the experimental results, 10 μM and 20 μM of ovatodiolide have inhibitory rings; the diameters of the inhibitory rings are 10±0.5 mm and 19±0 mm respectively. This proves that the minimum bacteriostatic concentration of ovatodiolide for inhibiting Helicobacter pylori is 10 μM.

TABLE 1
Minimum bacteriostatic concentrations for inhibiting
Helicobacter pylori type strain (H. pylori 26695)

| ovatodiolide concentrations (μM) | Inhibitory rings (mm) |
|---|---|
| 20 | 19 ± 0 |
| 10 | 10 ± 0.5 |
| 5 | — |
| 2.5 | — |
| 1.25 | — |
| 0 | — |

### Embodiment 2: Minimum Bactericidal Concentrations for Inhibiting Helicobacter pylori Type Strain and Multidrug-Resistant Strains Thereof

Different concentrations of ovatodiolide are prepared by using serial dilution method, and Helicobacter pylori type strain and multidrug-resistant gastric Helicobacter pylori (v633, v1354) separated from laboratory are cultivated for six hours respectively. Then, ovatodiolide is analyzed by using spread plate method to observe the number of colony growth, and analyze the minimum bactericidal concentrations of ovatodiolide for completely killing Helicobacter pylori type strain and multidrug-resistant strains. Based on the results on Table 2, the minimum bactericidal concentration of ovatodiolide for Helicobacter pylori type strain is 200 μM, and the minimum bactericidal concentrations for multidrug-resistant strains v633 and v1354 are 100 μM.

TABLE 2
Minimum bactericidal concentrations of ovatodiolide
for multidrug-resistant Helicobacter pylori

| strains | ovatodiolide concentrations (μM) |
|---|---|
| Hp reference strain 26695 | 200 |
| Hp v633 | 100 |
| Hp v1354 | 100 |

### Embodiment 3: Ovatodiolide for Inhibiting Protein Synthesis of Helicobacter pylori

The structure of ovatodiolide is similar to that of the medicines in antibiotics for inhibiting protein generation. In order to understand whether the mechanism of ovatodiolide for inhibiting Helicobacter Pylori is related to protein synthesis, let ovatodiolide and extracts of Escherichia coli (E. coli S30) to take effect for 10 minutes by using in vitro transcription/translation systems. Then, pGL3 luciferase is added for carrying out transcription and translation reaction for 2 hours. Finally, the reacted sample is added with luciferin for it to present color by using Dual-Luciferase Reporter Assay System. Then, microplate luminometer is used to determine the absorbance in order to find out whether ovatodiolide can inhibit protein synthesis.

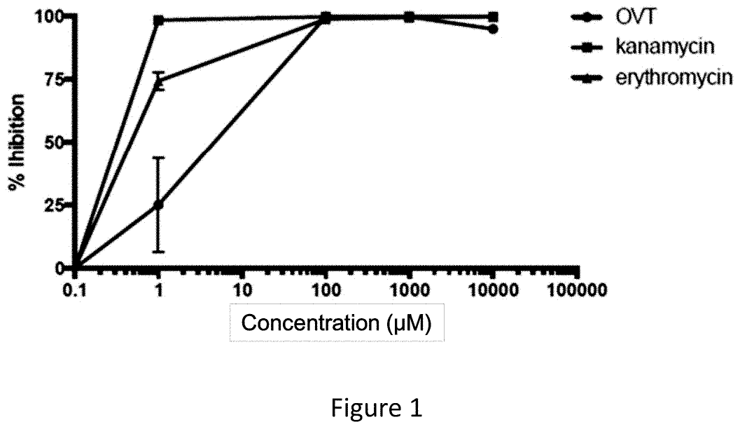

Among them, inhibition of 30S subunit using kanamycin and inhibition of 505 subunit using erythromycin are used as control groups. The results in FIG. 1 show that 10 μM of ovatodiolide can inhibit more than 50% of protein synthesis, and 1 μM of kanamycin and erythromycin can inhibit more than 75% of protein synthesis. Thus, the results show that the pharmaceutical mechanism of ovatodiolide is related to the inhibition of protein synthesis.

### Embodiment 4: Ovatodiolide for Inhibiting Initiation of Ribosomes of Helicobacter pylori

In this embodiment, the process of protein translation has three steps, namely, initiation, elongation and termination of ribosomes. In order to further analyze whether ovatodiolide is effected on ribosomes causing protein synthesis unable to proceed smoothly, western blot analysis is used to determine the content of protein RpsB in 30S ribosomal subunit. Helicobacter pylori is cultivated with ovatodiolide of different concentrations for 6 hours, and extract total cell lysates. Separate total cell lysates by using 10% SDS-PAGE, transfer protein using PVDF membrane, and carry out hybridization with protein using RpsB antibody. Then, detect RpsB antibody using anti-rabbit antibody; finally, a chromogenic reagent is added, and observe results by using the apparatus Azure C400 Biosystems.

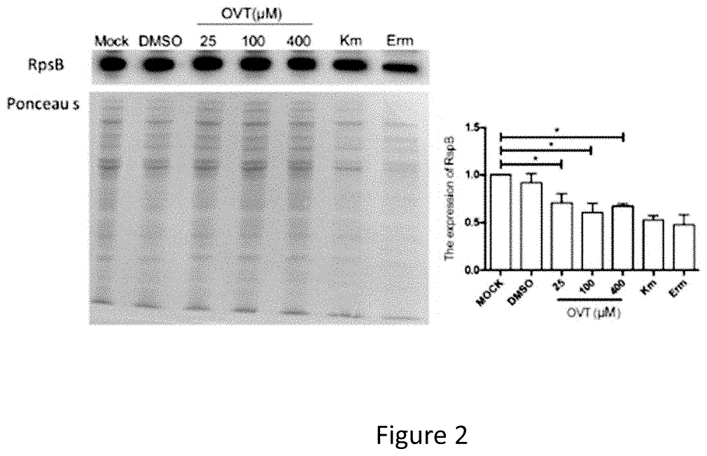

FIG. 2 is an expression of 30S ribosomal subunits RpsB analyzed by using western blot analysis. Wherein kanamycin (400 μM) and erythromycin (400 μM) are used as control groups. The results show that after protein RpsB and ovatodiolide took effect, the expression of protein reduces significantly, which is similar to the results in the control groups.

An effective concentration of ovatodiolide (molecular weight 328.4) in in vitro experiments for bacterial inhibition is from 10 μM to 1 mM. Using data of ovatodiolide (molecular weight 328.4) and approximately 5 liters of blood in an adult body of 65 kilograms, it is calculated that a daily effective dose for a human body is from 16 mg to 1642 mg; a preferred pharmaceutical concentration is from 50 μM to 500 μM, it is calculated that a daily preferred dose is from 82 mg to 821 mg; an optimal pharmaceutical concentration is from 100 μM to 200 μM, it is calculated that a daily optimal dose is from 164 mg to 329 mg.

As a conclusion from the above, an ovatodiolide of the present invention inhibits the protein synthesis by inhibiting the expression of 30S ribosomes RpsB, and further effectively inhibits the growth of Helicobacter pylori type strain and multidrug-resistant strains. The effective dose of ovatodiolide inhibiting gastric Helicobacter pylori is from 16 mg to 1642 mg; the preferred dose is from 82 mg to 821 mg; and the optimal dose is from 164 mg to 329 mg.

Note that the specification relating to the above embodiments should be construed as exemplary rather than as limitative of the present invention, with many variations and modifications being readily attainable by a person of average skill in the art without departing from the spirit or scope thereof as defined by the appended claims and their legal equivalents.

What is claimed is:

1. A method for inhibiting protein synthesis of gastric Helicobacter pylori and E. coli comprising:
administering a composition comprising an effective dose of ovatodiolide;
wherein the effective dose is between 16 mg and 1642 mg.

2. The method as claimed in claim 1, wherein the gastric Helicobacter pylori refers to multidrug-resistant gastric Helicobacter pylori or Helicobacter pylori type strain.

3. The method as claimed in claim 2, wherein the multidrug-resistant gastric Helicobacter pylori refers to HP v633 or HP v1354, and the Helicobacter pylori type strain is H. pylori 26695.

4. The method as claimed in claim 1, wherein the protein synthesis is carried out through the action of 30S ribosome RpsB.

5. (canceled)

6. The method as claimed in claim 1, wherein the effective dose is between 82 mg and 821 mg.

7. The method as claimed in claim 1, wherein the effective dose is between 164 mg and 329 mg.

8. The method as claimed in claim 1, wherein the composition further comprises pharmaceutically acceptable carrier, vehicle and thinner.

9. The method as claimed in claim 1, wherein a dosage form of the composition is selected from a group composed of solution, suspension, emulsion, powder, pastille, pellet, syrup, troche, tablet, chewing gum, jatex and capsule.

10. The method as claimed in claim 1, wherein the composition is further made into fluid milk products, concentrated milk, yogurt, sour milk, frozen yogurt, lactic acid bacteria-fermented beverages, milk powder, ice cream, cream cheeses, hard cheeses, soy milk, fermented soy milk, vegetable-fruit juices, juices, sports drinks, confectionery, jelly, candies, infant foods, health foods, animal feeds, Chinese medicinal herbs compositions and dietary supplements.
